(19) United States
(12) Patent Application Publication
(10) Pub. No.: US 2024/0095913 A1
(43) Pub. Date: Mar. 21, 2024

(54) APPARATUS AND METHOD FOR IMAGE TRANSFORMATION

(71) Applicant: RESEARCH COOPERATION FOUNDATION OF YEUNGNAM UNIVERSITY, Gyeongsangbuk-do (KR)

(72) Inventors: Gyu Sang CHOI, Daegu (KR); Kyeong Min PARK, Gyeongsangbuk-do (KR); Min Cheol CHANG, Daegu (KR)

(21) Appl. No.: 18/373,677

(22) Filed: Sep. 27, 2023

(30) Foreign Application Priority Data
Aug. 18, 2022 (KR) 10-2022-0103380

(51) Int. Cl. G06T 7/00; G06T 3/40; G06V 10/24; G06V 10/42; G06V 10/74

(52) U.S. Cl. CPC G06T 7/0012; G06T 3/40; G06V 10/242; G06V 10/42; G06V 10/761; G06T 2207/10081; G06T 2207/10088; G06T 2207/20084; G06T 2207/30016

(57) ABSTRACT

An apparatus for image transformation for transforming a first medical image into a second medical image based on an artificial neural network technology includes a preprocessing module that receives the first medical image and second medical image obtained by photographing the same part of a body using different photographing techniques and performs preprocessing on the first and second medical images, and an artificial neural network module that receives the preprocessed first medical image and second medical image, respectively, and transforms the first medical image into a second medical image.

### CROSS REFERENCE TO RELATED APPLICATIONS AND CLAIM OF PRIORITY

This application claims the benefit under 35 USC § 119 of Korean Patent Application No. 10-2022-0103380, filed on Aug. 18, 2022, in the Korean Intellectual Property Office, the entire disclosure of which is incorporated herein by reference for all purposes.

### BACKGROUND

### 1. Field

Embodiments of the present disclosure relate to an image transformation technology for a medical image.

### 2. Description of Related Art

Recently, with the development of deep learning technology, various attempts using deep learning technology are being made in a medical field. For example, various research using deep learning technology are being conducted based on medical images such as X-ray, computer tomography (CT), and magnetic resonance imaging (MRI).

Here, the magnetic resonance imaging (MRI) is a tomography method using the principles of nuclear magnetic resonance. Specifically, hydrogen molecules inside the human body undergo precession at a specific frequency, and when electromagnetic waves of the same frequency are applied to the hydrogen molecules, the hydrogen molecules resonate and absorb energy. An MRI image is an image reconstructed by a computer by measuring the phases when the hydrogen molecules release the absorbed energy.

The computer tomography (CT) is a photographing method that restores cross-sectional images using X-ray images photographed from various directions. A CT image has a feature of clearly illustrating a hard part of the human body. Even when MTI and CT photograph the same part of the body, they share a certain feature but obtain images with different properties.

### SUMMARY

Embodiments of the present disclosure are intended to provide a new medical image transformation technology.

According to an embodiment of the present disclosure, there is provided an apparatus for image transformation is an apparatus for image transformation for transforming a first medical image into a second medical image based on an artificial neural network technology, the apparatus including a preprocessing module that receives the first medical image and second medical image obtained by photographing the same part of a body using different photographing techniques and performs preprocessing on the first and second medical images, and an artificial neural network module that receives the preprocessed first medical image and second medical image, respectively, and transforms the first medical image into a second medical image.

The preprocessing module may rotate the first medical image and the second medical image to align central axes thereof and resize an image size of each of the first and second medical images to a preset size.

The first medical image may be a computer tomography (CT) image, and the second medical image may be a magnetic resonance imaging (MRI) image.

The artificial neural network module may include a first artificial neural network model trained to generate an intermediate transformed image by receiving the CT image and a second artificial neural network model trained to generate an MRI image by receiving the intermediate transformed image generated by the first artificial neural network model.

The first artificial neural network model may include a first generator that receives the CT image and generates the intermediate transformed image from the input CT image and a first discriminator that receives the intermediate transformed image and an original MRI image, respectively, and compares the intermediate transformed image and the original MRI image to output a classification value.

The first generator may generate the intermediate transformed image so that a global feature of boundaries of respective regions for internal parts of the body is revealed in the CT image and a characteristic of the MRI image is reflected in the intermediate transformed image.

The first generator may include a down block that downsamples the CT image, a plurality of residual blocks sequentially connected to an output stage of the down block and provided to extract a feature by performing dilated convolution on the CT image, and an up block that generates the intermediate transformed image by up-sampling the feature extracted from the residual block.

The plurality of residual blocks may include a plurality of block groups, and the block groups may be provided so that a dilation coefficient according to the dilated convolution becomes smaller as the block group approaches the up block.

The first discriminator may include a first concatenation layer that generates a concatenated image by concatenating the intermediate transformed image output from the first generator and the original MR image, a parallel block connected in parallel with the first concatenation layer and including a plurality of convolution layers and a pooling layer each extracting a feature from the concatenated image, a second concatenation layer that generate a concatenated vector by concatenating respective features extracted from the parallel block, and a discrimination layer that outputs a classification value based on the concatenated vector.

The second artificial neural network model may include a second generator that receives the intermediate transformed image from the first artificial neural network model and generates an MRI image from the intermediate transformed image, and a second discriminator that receives the generated MRI image and the original MRI image, respectively, and compares the generated MRI image and the original MRI image to output a classification value.

The second generator may generate the MRI image so that a regional feature existing inside respective regions of the internal parts of the body is revealed in the intermediate transformed image.

The second discriminator may include a concatenation layer that generates a concatenated image by concatenating the generated MRI image and the original MRI image, a plurality of convolution layers that extract a feature vector from the concatenated image and are sequentially connected to each other, and a discrimination layer that outputs a classification value based on the extracted feature vector.

The first artificial neural network model may generate the intermediate transformed image so that a global feature of boundaries of respective regions for internal parts of the body is revealed in the CT image and a characteristic of the MRI image is reflected in the intermediate transformed image, and the second artificial neural network model may generate the MRI image so that a regional feature existing inside respective regions of the internal parts of the body is revealed in the intermediate transformed image.

According to another embodiment of the present disclosure, there is provided a method for image transformation performed on a computing device including a first artificial neural network model and a second artificial neural network model, the method including training the first artificial neural network model to generate an intermediate transformed image by receiving a CT image and training the second artificial neural network model to generate an MRI image by receiving the intermediate transformed image generated by the first artificial neural network model.

The training of the first artificial neural network model may include receiving, by a first generator the CT image and generating the intermediate transformed image from the input CT image and receiving, by a first discriminator, the intermediate transformed image and an original MRI image, respectively, and comparing the intermediate transformed image and the original MRI image to output a classification value.

The training of the second artificial neural network model may include receiving, by a second generator, the intermediate transformed image from the first artificial neural network model and generating an MRI image from the intermediate transformed image, and receiving, by a second discriminator, the generated MRI image and the original MRI image, respectively, and comparing the generated MRI image and the original MRI image to output a classification value.

The first artificial neural network model may be trained to generate the intermediate transformed image so that a global feature of boundaries of respective regions for internal parts of the body is revealed in the CT image and a characteristic of the MRI image is reflected in the intermediate transformed image, and the second artificial neural network model may be trained to generate the MRI image so that a regional feature existing inside respective regions of the internal parts of the body is revealed in the intermediate transformed image.

According to the disclosed embodiments, the intermediate transformed image in which the global feature of internal parts of the body is revealed is generated from the CT image through the first artificial neural network model and the MRI image in which a regional feature within each region of the internal parts of the body is revealed is generated from the intermediate transformed images through the second artificial neural network model, thereby transforming the CT image into the MRI image more accurately.

### BRIEF DESCRIPTION OF THE DRAWINGS

FIG. 1 is a block diagram illustrating an apparatus for image transformation according to an embodiment of the present disclosure.

FIG. 2 is a diagram illustrating a process of pre-processing a raw CT image by a pre-processing module according to an embodiment of the present disclosure.

FIG. 3 is a diagram illustrating a configuration of an artificial neural network module according to an embodiment of the present disclosure.

FIG. 4 is a diagram illustrating a structure of a first generator according to an embodiment of the present disclosure.

FIGS. 5A to 5C illustrate dilated convolution filters having dilation coefficients of 1, 2, and 3.

FIG. 6 is a diagram illustrating a structure of a first discriminator according to an embodiment of the present disclosure.

FIG. 7 is a diagram illustrating a structure of a second discriminator according to an embodiment of the present disclosure.

FIGS. 8A to 8C are diagrams illustrating a CT image (original), an MRI image (original), and an intermediate transformed image according to an embodiment of the present disclosure.

FIG. 9 is a diagram illustrating the CT image (original), the MRI image (original), and a generated MR image according to an embodiment of the present disclosure.

FIG. 10 is a block diagram for illustratively describing a computing environment including a computing device suitable for use in exemplary embodiments.

### DETAILED DESCRIPTION

Hereinafter, a specific embodiment of the present disclosure will be described with reference to the drawings. The following detailed description is provided to aid in a comprehensive understanding of the methods, apparatus and/or systems described herein. However, this is illustrative only, and the present disclosure is not limited thereto.

In describing the embodiments of the present disclosure, when it is determined that a detailed description of related known technologies may unnecessarily obscure the subject matter of the present disclosure, a detailed description thereof will be omitted. In addition, terms to be described later are terms defined in consideration of functions in the present disclosure, which may vary according to the intention or custom of users or operators. Therefore, the definition should be made based on the contents throughout this specification. The terms used in the detailed description are only for describing embodiments of the present disclosure, and should not be limiting. Unless explicitly used otherwise, expressions in the singular form include the meaning of the plural form. In this description, expressions such as “comprising” or “including” are intended to refer to certain features, numbers, steps, actions, elements, some or combination thereof, and it is not to be construed to exclude the presence or possibility of one or more other features, numbers, steps, actions, elements, some or combinations thereof, other than those described.

Further, terms such as first, second, etc., may be used to describe various components, but the components are not limited by the terms. The above terms may be used for the purpose of distinguishing one component from another. For example, a first component may be termed a second component, and similarly, a second component may be termed a first component, without departing from the scope of the present disclosure.

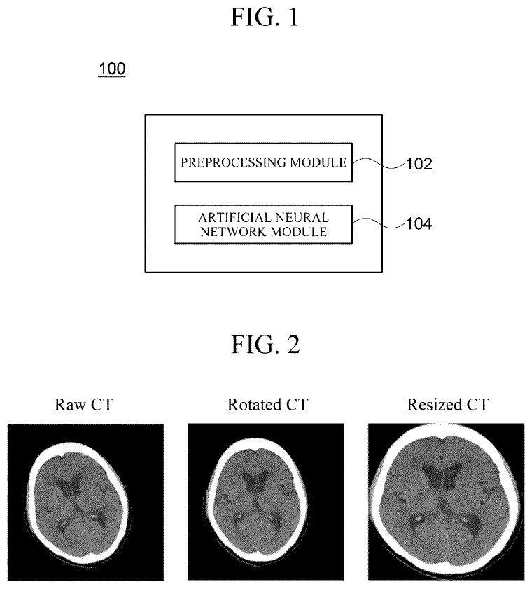

FIG. 1 is a block diagram illustrating an apparatus for image transformation according to an embodiment of the present disclosure.

Referring to FIG. 1, an apparatus for image transformation 100 may include a preprocessing module 102 and an artificial neural network module 104.

The apparatus for image transformation 100 may be an apparatus for transforming a first medical image into a second medical image based on artificial neural network technology. Here, the first medical image and the second medical image may be images obtained by different photographing techniques. In an exemplary embodiment, the first medical image may be a computer tomography (CT) image, and the second medical image may be a magnetic resonance imaging (MRI) image.

In this case, the apparatus for image transformation 100 may be an apparatus for transforming the CT image into the MRI image. Hereinafter, the case where the first medical image is a CT image and the second medical image is an MRI image will be described as an example, but is not limited thereto, and the first medical image may be the MRI image and the second medical image may be the CT image.

The preprocessing module 102 can preprocess each of an input raw CT image and raw MRI image. Here, the raw CT image is an image photographed by computer tomography technique for a part of the body (e.g., the brain part). The Raw MRI image is an image in which the same part as the part photographed in the CT image is photographed for the same person by the magnetic resonance imaging technique. The preprocessed CT image and MRI image (original) can be one data set for training the artificial neural network module 104.

The preprocessing module 102 can rotate the raw CT image and the raw MRI image to align the central axes thereof. Further, the preprocessing module 102 may remove the background from the raw CT image and the raw MRI image and resize each of image sizes thereof to a preset size.

FIG. 2 is a diagram illustrating a process of pre-processing the raw CT image by the pre-processing module 102 according to an embodiment of the present disclosure. The raw MRI image can also be preprocessed using the same process. Referring to FIG. 2, if the central axis of an object (e.g., a brain part of the body) photographed in the raw CT image is distorted, the preprocessing module 102 may rotate the central axis so that the central axis is aligned. Additionally, the preprocessing module 102 may remove the background from the raw CT image and resize the image size thereof to a preset size (e.g., 256×256). The preprocessing module 102 can transmit the preprocessed CT image and MRI image to the artificial neural network module 104.

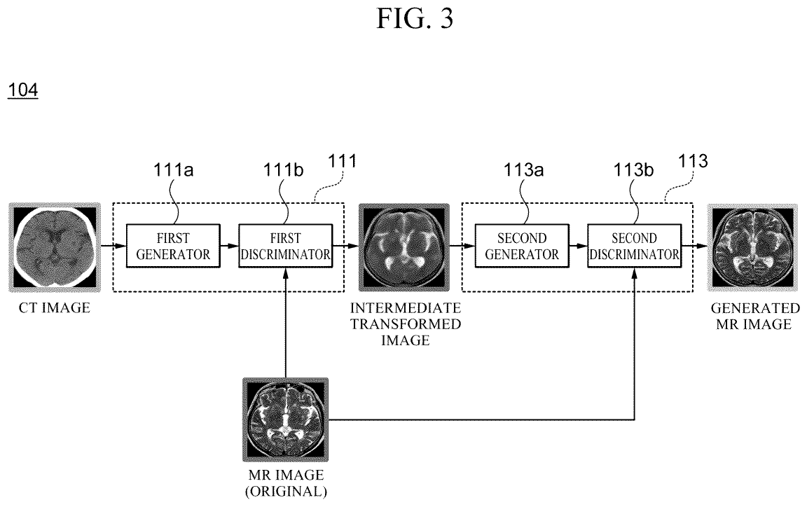

FIG. 3 is a diagram illustrating a configuration of the artificial neural network module 104 according to an embodiment of the present disclosure. Referring to FIG. 3, the artificial neural network module 104 may include a first artificial neural network model 111 and a second artificial neural network model 113. The artificial neural network module 104 can receive a CT image and transform the input CT image into an MRI image. In this time, the artificial neural network module 104 may compare the transformed MRI image with the MRI image (original) received from the preprocessing module 102 to minimize the difference therebetween.

The first artificial neural network model 111 may include a first generator 111a and a first discriminator 111b. That is, the first artificial neural network model 111 may be configured as a generative adversarial network.

The first generator 111a may receive a CT image and generate an intermediate transformed image from the CT image. Specifically, the first generator 111a may be an artificial neural network that is trained to generate an intermediate transformed image containing a feature of boundaries of respective regions for internal parts of the body (hereinafter, may be referred to as a global feature) on the CT image from the CT image.

For example, if the CT image is an image of the brain part of the body, the first generator 111a can generate an intermediate transformed image that reveals the boundaries of respective brain parts, such as gray matter, white matter, basal ganglia, and ventricle, in the CT image.

When the artificial neural network module 104 receives a CT image and transforms it into an MRI image, the first artificial neural network model 111 may be a model for generating an intermediate transformed image, which is an intermediate image before transformation from the CT image to the MRI image. In this case, the first generator 111a may receive the CT image and generate the intermediate transformed image that reveals the global feature of internal parts of the body (i.e., feature of the overall shape of respective regions of the internal parts of the body) in the CT images.

And, when generating the intermediate transformed image from the CT image, the first artificial neural network model 111 can reveal the global feature of internal parts of the body in the intermediate transformed image and reveal a characteristic of the MRI image. That is, in the case of the CT image, hard parts of the body (e.g., bones, etc.) appear in white (pixel value close to 255), soft parts of the body (e.g., nerve bundles, blood, soft tissue, etc.) appear in black (pixel value close to 0), and medium-hard parts (e.g., cartilage, muscle, etc.) of the body appear in gray. On the other hand, in the case of the MRI image, hard parts of the body appear in black because they have little moisture, soft parts of the body (e.g., cerebrospinal fluid, ventricles, etc.) appear in white because they have a lot of moisture, and other parts appear in gray.

The first artificial neural network model 111 allows a portion represented in white in the CT image to be represented in black in the intermediate transformed image, allows a portion represented in black in the CT image to be represented in black in the intermediate transformed image, and allows a portion represented in gray in the CT image to be represented in gray in the intermediate transformed image, thereby capable of allowing the intermediate transformed image to reflect the characteristic of the MRI image.

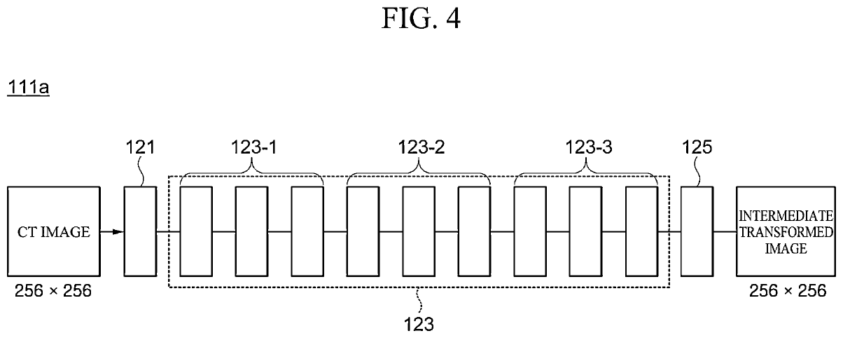

FIG. 4 is a diagram illustrating the structure of the first generator 111a according to an embodiment of the present disclosure. Referring to FIG. 4, the first generator 111a may include a down block 121, a residual block 123, and an up block 125.

The down block 121 can downsample the input CT image. A plurality of residual blocks 123 may be sequentially connected to each other between the down block 123 and the up block 125. In an exemplary embodiment, each residual block 123 may be provided to perform dilated convolution on the downsampled CT image. That is, the residual block 123 can perform convolution using a dilated convolution filter.

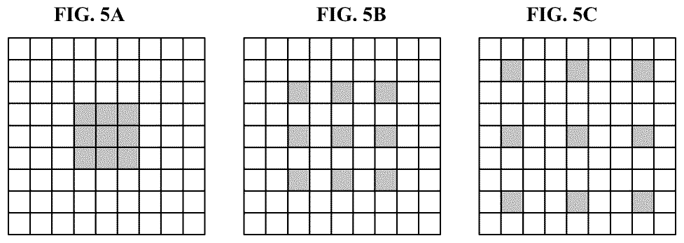

The dilated convolution filter maintains the same number of filter parameters by leaving a gap between the pixels of the filter, but at the same time increases a receptive field, thereby resulting in an effect similar to using a large-sized filter. FIGS. 5A to 5C illustrate dilated convolution filters, and cases where the dilation coefficients are 0, 1, and 2 are illustrated in FIGS. 5A to 5C, respectively. That is, the dilation coefficient can be determined depending on how many gaps there are between pixels in the filter.

The residual block 123 may be configured with three block groups 123-1, 123-2, and 123-2, and a first block group 123-1 may have a dilation coefficient (or dilation rate) of 5, a second block group 123-2 may have a dilation coefficient of 3, and a third block group may have a dilation coefficient of 5. That is, the dilation coefficient of the block group may be provided to become smaller as it approaches the up block 125. The residual block 123 performs dilation convolution to extract a feature from the CT image.

The up block 125 can generate the intermediate transformed image by upsampling the feature extracted from the residual block 123.

In this way, by performing dilation convolution on the downsampled CT image, it is possible to extract the global feature while intentionally ignoring a detailed feature (local feature) in the CT image.

The first discriminator 111b may receive the intermediate transformed image from the first generator 111a and an MRI image (original) from the preprocessing module 102. The first discriminator 111b may be trained to compare the intermediate transformed image and the MRI image and classify it as True if the intermediate transformed image is the same as the MRI image, and classify it as Fake if the intermediate transformed image is not the same as the MRI image. The first discriminator 111b may be designed to classify, as True, a result that clearly reveals the global feature of internal parts of the body in the intermediate transformed image, and classify as Fake otherwise.

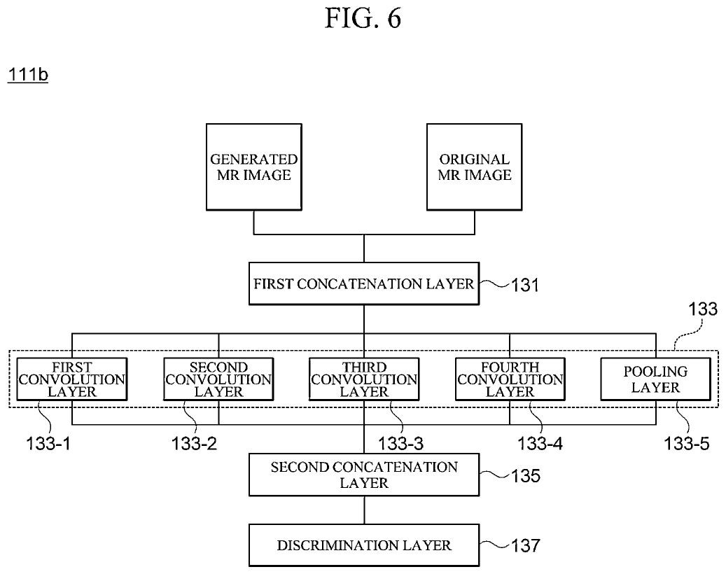

FIG. 6 is a diagram illustrating a structure of the first discriminator 111b according to an embodiment of the present disclosure. Referring to FIG. 6, the first discriminator 111b may include a first concatenation layer 131, a parallel block 133, a second concatenation layer 135, and a discrimination layer 137.

The first concatenation layer 131 can generate a concatenated image by concatenating the intermediate transformed image output from the first generator 111a and the MRI image (original). The first concatenation layer 131 can generate the concatenated image by concatenating a value of each pixel of the intermediate transformed image with a value of each pixel of the MRI image (original). In this case, the size of the intermediate transformed image and the size of the MRI image (original) may be the same.

The parallel block 133 is for extracting the feature from the concatenated image, and may include a plurality of convolution layers 133-1 to 133-4 and a pooling layer 133-5. The plurality of convolution layers 133-1 to 133-4 and the pooling layer 133-5 are connected in parallel, and each of the convolution layers 133-1 to 133-4 and the pooling layer 133-5 receives the first concatenated image and extracts the feature.

Here, each of the first convolution layer 133-1 to the third convolution layer 133-3 can extract the feature from the concatenated image by performing dialed convolution. The first convolution layer 133-1 to the third convolution layer 133-3 may have different dilation coefficients. For example, the first convolution layer 133-1 may have a dilation coefficient of 18, the second convolution layer 133-2 may have a dilation coefficient of 12, and the third convolution layer 133-3 may have a dilation coefficient of 6, but is not limited thereto. The first convolution layer 133-1 to the third convolution layer 133-3 may perform dilated convolution using a filter having a size of 3×3.

The fourth convolution layer 133-4 may be a 1×1 convolution layer. That is, the fourth convolution layer 133-4 can perform convolution on the concatenated image using a filter having a size of 1×1. Further, the pooling layer 133-5 can extract the feature by performing global average pooling on the concatenated images.

The second concatenation layer 135 can generate a concatenated vector by concatenating respective features extracted from the parallel block 133. That is, the second concatenation layer 135 can generate a concatenated vector by concatenating the features extracted from the plurality of convolution layers 133-1 to 133-4 and the pooling layer 133-5.

The discrimination layer 137 may output a classification value (True or Fake) based on the concatenated vector output from the second concatenation layer 135. For example, the discrimination layer 137 may output the classification value based on a ratio of 0 and 1 included in the concatenated vector, but is not limited thereto.

The second artificial neural network model 113 may include a second generator 113a and a second discriminator 113b. That is, the second artificial neural network model 113 may be configured as the generative adversarial network. The second artificial neural network model 113 is an independent model from the first artificial neural network model 111, and may be a model trained independently from the first artificial neural network model 111.

The second generator 113a may receive the intermediate transformed image and generate the MRI image from the intermediate transformed image. The second generator 113a is configured with an artificial neural network separate from the first generator 111a, and can generate the MRI image by receiving the intermediate transformed image output from the first generator 111a.

The second generator 113a can generate the MRI image by including the detailed feature (hereinafter referred to as a regional feature) existing inside respective regions for internal parts of the body in the intermediate transformed image.

For example, when the first generator 111a generates an intermediate transformed image so as to reveal the boundaries of respective regions such as gray matter, white matter, basal ganglia, ventricle, etc., the second generator 113a may generate an MRI image by revealing regional features such as vascular tissue, vascular patterns or shapes, or wrinkles (e.g. sulcus, gyms, etc.) within respective regions such as gray matter, white matter, basal ganglia, and ventricle.

In an exemplary embodiment, the structure of the second generator 113a may be the same as the structure of the first generator 111a. That is, the second generator 113a may also be configured with a down block, a residual block, and an up block. However, since the second generator 113a is a separate artificial neural network from the first generator 111a and does not share the training parameters of the first generator 111a, unlike the first generator 111a, the second generator 113a generates the MRI image by including the regional feature existing inside respective regions of the internal parts of the body in the intermediate transformed image.

The second discriminator 113b may receive the MRI image generated from the second generator 113a and the MRI image (original) from the preprocessing module 102.

The second discriminator 113b may be trained to compare the generated MRI image and the MRI image (original) and classify it as True if the generated MRI image is the same as the MRI image (original), and classify it as Fake if the generated MRI image is not the same as the MRI image (original). In this case, the second discriminator 113b may be designed to classify, as True, a result that clearly reveals the regional feature within respective regions of the internal parts of the body in the generated MRI image, and classify it as Fake otherwise.

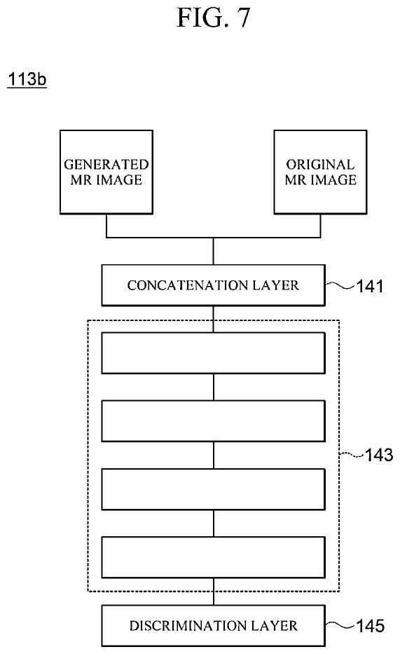

FIG. 7 is a diagram illustrating a structure of the second discriminator 113b according to an embodiment of the present disclosure. Referring to FIG. 7, the second discriminator 113b may include a concatenation layer 141, a plurality of convolution layers 143, and a discrimination layer 145.

The concatenation layer 141 can generate a concatenated image by concatenating the generated MRI image and the original MRI image. The concatenation layer 141 can generate the concatenated image by concatenating the value of each pixel of the generated MRI image with the value of each pixel of the original MRI image. In this case, the size of the generated MRI image and the size of the original MRI image may be the same.

The plurality of convolution layers 143 can extract a feature (i.e., feature vector) from the concatenated image. The plurality of convolution layers 143 may be connected sequentially (connected in series). Each of the plurality of convolution layers 143 can sequentially perform convolution to extract the feature from the concatenated image. Here, four convolution layers are shown, but is not limited thereto.

The discrimination layer 145 may output a classification value (True or Fake) based on the feature vector extracted from the plurality of convolution layers 143.

According to the disclosed embodiment, the intermediate transformed image revealing the global feature of internal parts of the body is generated from the CT image through the first artificial neural network model and the MRI image revealing a regional feature within respective regions of the internal parts of the body is generated from the intermediate transformed images through the second artificial neural network model, thereby capable of transforming the CT image into the MRI image more accurately.

That is, since the CT image and the MRI image are gray-scale images and have black-and-white information, their features are not clearly extracted compared to 3-channel color information (RGB). Accordingly, an intermediate transformed image is generated through the first artificial neural network model so as to reveal the rough outline of internal parts of the body while ignoring detailed features in the CT image, and the detailed feature (regional feature) are revealed in the intermediate transformation image through the second artificial neural network model, thereby generating an MRI image.

In this specification, a module may mean a functional and structural combination of hardware for implementing the technical idea of the present disclosure and software for driving the hardware. For example, the “module” may mean a logical unit of a predetermined code and hardware resources for executing the predetermined code, and does not necessarily mean a physically connected code or a single type of hardware.

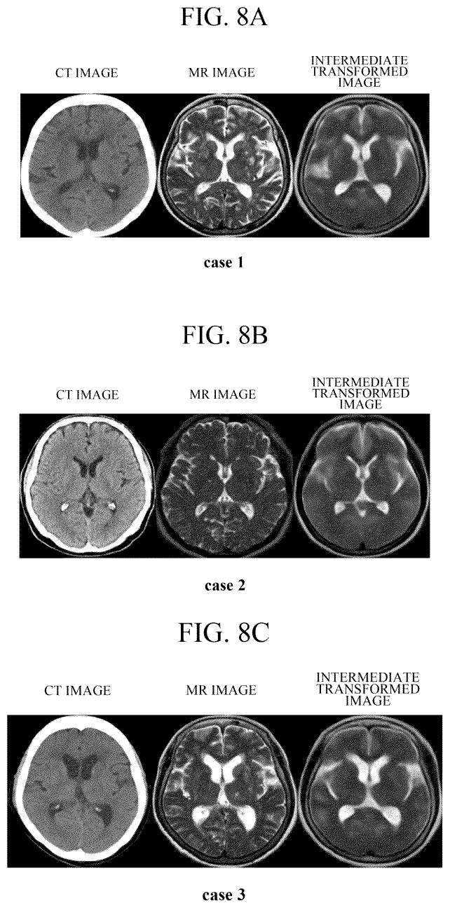

FIGS. 8A to 8C are diagrams illustrating the CT image (original), the MRI image (original), and the intermediate transformed image according to an embodiment of the present disclosure. Referring to FIGS. 8A to 8C, it can be seen that the intermediate transformed image has the characteristic of the MRI image while revealing the boundaries of respective regions of the internal parts of the body.

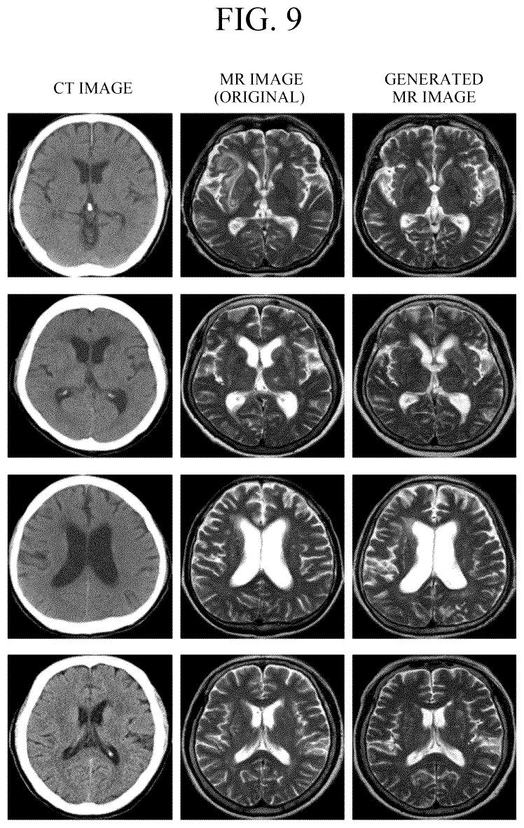

FIG. 9 is a diagram illustrating the CT image (original), the MRI image (original), and the generated MR image according to an embodiment of the present disclosure. Referring to FIG. 9, it can be seen that the generated MR image is quite similar to the original MR image.

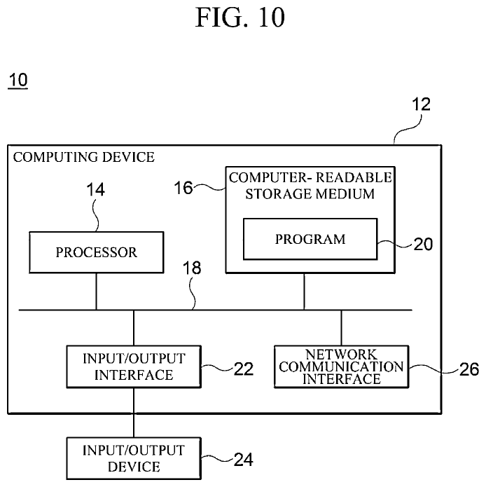

FIG. 10 is a block diagram for illustratively describing a computing environment 10 including a computing device suitable for use in exemplary embodiments. In the illustrated embodiment, respective components may have different functions and capabilities other than those described below, and may include additional components in addition to those described below.

The illustrated computing environment 10 includes a computing device 12. In an embodiment, the computing device 12 may be the apparatus for image transformation 100.

The computing device 12 includes at least one processor 14, a computer-readable storage medium 16, and a communication bus 18. The processor 14 may cause the computing device 12 to operate according to the exemplary embodiment described above. For example, the processor 14 may execute one or more programs stored on the computer-readable storage medium 16. The one or more programs may include one or more computer-executable instructions, which, when executed by the processor 14, may be configured so that the computing device 12 performs operations according to the exemplary embodiment.

The computer-readable storage medium 16 is configured so that the computer-executable instruction or program code, program data, and/or other suitable forms of information are stored. A program 20 stored in the computer-readable storage medium 16 includes a set of instructions executable by the processor 14. In one embodiment, the computer-readable storage medium 16 may be a memory (volatile memory such as a random access memory, non-volatile memory, or any suitable combination thereof), one or more magnetic disk storage devices, optical disk storage devices, flash memory devices, other types of storage media that are accessible by the computing device 12 and capable of storing desired information, or any suitable combination thereof.

The communication bus 18 interconnects various other components of the computing device 12, including the processor 14 and the computer-readable storage medium 16.

The computing device 12 may also include one or more input/output interfaces 22 that provide an interface for one or more input/output devices 24, and one or more network communication interfaces 26. The input/output interface 22 and the network communication interface 26 are connected to the communication bus 18. The input/output device 24 may be connected to other components of the computing device 12 through the input/output interface 22. The exemplary input/output device 24 may include a pointing device (such as a mouse or trackpad), a keyboard, a touch input device (such as a touch pad or touch screen), a speech or sound input device, input devices such as various types of sensor devices and/or photographing devices, and/or output devices such as a display device, a printer, a speaker, and/or a network card. The exemplary input/output device 24 may be included inside the computing device 12 as a component configuring the computing device 12, or may be connected to the computing device 12 as a separate device distinct from the computing device 12.

Although representative embodiments of the present disclosure have been described in detail, a person skilled in the art to which the present disclosure pertains will understand that various modifications may be made thereto within the limits that do not depart from the scope of the present disclosure. Therefore, the scope of rights of the present disclosure should not be limited to the described embodiments, but should be defined not only by claims set forth below but also by equivalents to the claims.

1: An apparatus for image transformation for transforming a first medical image into a second medical image based on an artificial neural network technology, the apparatus comprising:
a preprocessing module configured to receive the first medical image and second medical image obtained by photographing the same part of a body using different photographing techniques and perform preprocessing on the first and second medical images; and
an artificial neural network module configured to receive the preprocessed first medical image and second medical image, respectively, and transform the first medical image into a second medical image.

2: The apparatus of claim 1, wherein the preprocessing module is configured to rotate the first medical image and the second medical image to align central axes thereof and resize an image size of each of the first and second medical images to a preset size.

3: The apparatus of claim 1, wherein the first medical image is a computer tomography (CT) image, and
the second medical image is a magnetic resonance imaging (MRI) image.

4: The apparatus of claim 3, wherein the artificial neural network module includes:
a first artificial neural network model trained to generate an intermediate transformed image by receiving the CT image; and
a second artificial neural network model trained to generate an MRI image by receiving the intermediate transformed image generated by the first artificial neural network model.

5: The apparatus of claim 4, wherein the first artificial neural network model includes:
a first generator configured to receive the CT image and generate the intermediate transformed image from the input CT image; and
a first discriminator configured to receive the intermediate transformed image and an original MRI image, respectively, and compare the intermediate transformed image and the original MRI image to output a classification value.

6: The apparatus of claim 5, wherein the first generator is configured to generate the intermediate transformed image so that a global feature of boundaries of respective regions for internal parts of the body is revealed in the CT image and a characteristic of the MRI image is reflected in the intermediate transformed image.

7: The apparatus of claim 5, wherein the first generator includes
a down block configured to downsample the CT image;
a plurality of residual blocks sequentially connected to an output stage of the down block and provided to extract a feature by performing dilated convolution on the CT image; and
an up block configured to generate the intermediate transformed image by up-sampling the feature extracted from the residual block.

8: The apparatus of claim 7, wherein the plurality of residual blocks include a plurality of block groups, and
the block groups are provided so that a dilation coefficient according to the dilated convolution becomes smaller as the block group approaches the up block.

9: The apparatus of claim 7, wherein the first discriminator includes:
a first concatenation layer configured to generate a concatenated image by concatenating the intermediate transformed image output from the first generator and the original MR image;
a parallel block connected in parallel with the first concatenation layer and including a plurality of convolution layers and a pooling layer each extracting a feature from the concatenated image;
a second concatenation layer configured to generate a concatenated vector by concatenating each feature extracted from the parallel block; and
a discrimination layer configured to output a classification value based on the concatenated vector.

10: The apparatus of claim 6, wherein the second artificial neural network model includes:
a second generator configured to receive the intermediate transformed image from the first artificial neural network model and generate an MRI image from the intermediate transformed image; and
a second discriminator configured to receive the generated MRI image and the original MRI image, respectively, and compare the generated MRI image and the original MRI image to output a classification value.

11: The apparatus of claim 10, wherein the second generator is configured to generate the MRI image so that a regional feature existing inside respective regions of the internal parts of the body are revealed in the intermediate transformed image.

12: The apparatus of claim 10, wherein the second discriminator includes:
a concatenation layer configured to generate a concatenated image by concatenating the generated MRI image and the original MRI image;
a plurality of convolution layers that are configured to extract a feature vector from the concatenated image and are sequentially connected to each other; and
a discrimination layer configured to output a classification value based on the extracted feature vector.

13: The apparatus of claim 4, wherein the first artificial neural network model is configured to generate the intermediate transformed image so that a global feature of boundaries of respective regions for internal parts of the body is revealed in the CT image and a characteristic of the MRI image is reflected in the intermediate transformed image, and
the second artificial neural network model is configured to generate the MRI image so that a regional feature existing inside respective regions of the internal parts of the body is revealed in the intermediate transformed image.

14: A method for image transformation performed on a computing device including a first artificial neural network model and a second artificial neural network model, the method comprising:
training the first artificial neural network model to generate an intermediate transformed image by receiving a CT image; and
training the second artificial neural network model to generate an MRI image by receiving the intermediate transformed image generated by the first artificial neural network model.

15: The method of claim 14, wherein the training of the first artificial neural network model includes:
receiving, by a first generator the CT image and generating the intermediate transformed image from the input CT image; and
receiving, by a first discriminator, the intermediate transformed image and an original MRI image, respectively, and comparing the intermediate transformed image and the original MRI image to output a classification value.

16: The method of claim 15, wherein the training of the second artificial neural network model includes:
receiving, by a second generator, the intermediate transformed image from the first artificial neural network model and generating an MRI image from the intermediate transformed image; and
receiving, by a second discriminator, the generated MRI image and the original MRI image, respectively, and comparing the generated MRI image and the original MRI image to output a classification value.

17: The method of claim 14, wherein the first artificial neural network model is trained to generate the intermediate transformed image so that a global feature of boundaries of respective regions for internal parts of the body is revealed in the CT image and a characteristic of the MRI image is reflected in the intermediate transformed image, and
the second artificial neural network model is trained to generate the MRI image so that a regional feature existing inside respective regions of the internal parts of the body is revealed in the intermediate transformed image.

18: A computer program stored in a non-transitory computer readable storage medium, the computer program including one or more instructions, which, when executed by a computing device having one or more processors and including a first artificial neural network model and a second artificial neural network model, cause the computing device to execute:
training the first artificial neural network model to generate an intermediate transformed image by receiving a CT image; and
training the second artificial neural network model to generate an MRI image by receiving the intermediate transformed image generated by the first artificial neural network model.
